SEQUENCE LISTING (11 sequences)

SEQ ID NO: 1 - DNA, 29 bp, Artificial sequence
  Synthetic oligonucleotide
ggtttccaaggatcctctcaaggtatgtc

SEQ ID NO: 2 - DNA, 26 bp, Artificial sequence
  Synthetic oligonucleotide
ccaccaaacatccatgggaattctgc

SEQ ID NO: 3 - DNA, 85 bp, Artificial sequence
  Synthetic oligonucleotide
agctggatccatggatgcagaattccgacatgactcaggatatgaagttcatcatcaaaaattggtgttctttgcagaagatgtg

SEQ ID NO: 4 - DNA, 85 bp, Artificial sequence
  Synthetic oligonucleotide
attaagatctcgctatgacaacaccgcccaccatgagtccaatgattgcacctttgtttgaacccacatcttctgcaaagaacac

SEQ ID NO: 5 - DNA, 28 bp, Artificial sequence
  Synthetic oligonucleotide
tctcacggccggtctttgaacgactttc

SEQ ID NO: 6 - DNA, 26 bp, Artificial sequence
  Synthetic oligonucleotide
ccaccaaacatccatgggaattctgc

SEQ ID NO: 7 - DNA, 37 bp, Artificial sequence
  Synthetic oligonucleotide
catcatcaaaaattggtgaccactgcagaagatgtgg

SEQ ID NO: 8 - DNA, 37 bp, Artificial sequence
  Synthetic oligonucleotide
ccacatcttctgcagtggtcaccaatttttgatgatg

SEQ ID NO: 9 - DNA, 42 bp, Artificial sequence
  Synthetic oligonucleotide
gggttcaaacaaaggtgcaccaattggactcatggtgggcgg

SEQ ID NO: 10 - DNA, 42 bp, Artificial sequence
  Synthetic oligonucleotide
ccgcccaccatgagtccaattggtgcacctttgtttgaaccc

SEQ ID NO: 11 - protein, 42 aa, Homo sapiens [sequence omitted]

(19) United States
(12) Patent Application Publication
(10) Pub. No.: US 2010/0196932 A1
(43) Pub. Date: Aug. 5, 2010

(54) Yeast Reporter System

(75) Inventors: Susan W. Liebman, Glenview, IL (US); Sviatoslav N. Bagriantsev, Wood Dale, IL (US)

Correspondence Address: JANE MASSEY LICATA, ESQ.;LICATA & TYRRELL P.C., 66 E. MAIN STREET, MARLTON, NJ, 08053, US

(73) Assignee: THE BOARD OF TRUSTEES OF THE UNIVERSITY OF ILLINOIS, URBANA, IL (US)

(21) Appl. No.: 12/281,021

(22) PCT Filed: Mar. 2, 2007
(86) PCT No.: PCT/US07/63178

§ 371 Date: Dec. 22, 2008

Related U.S. Application Data
(60) Provisional application No. 60/778,491, filed on Mar. 2, 2006.

(51) Int. Cl. G01N 33/569

(52) U.S. Cl. 435/7.31

(57) ABSTRACT

Cell-based screens for compounds that inhibit the accumulation of amyloids are described. For example, the herein described screens may be used to assay the formation, or inhibition, of pre-amyloid or amyloid aggregates and/or oligomers. Agents have been identified that interfere with, or inhibit, the oligomerization of the major component of amyloid plaques; a 42 amino acid long Aβ42 peptide product of proteolytic processing of the APP protein.

### CROSS REFERENCE TO RELATED APPLICATIONS

This application claims the benefit of U.S. Provisional Application 60/778,491, filed on Mar. 2, 2006.

### GOVERNMENT RIGHTS

This work was supported by NIH Grant No. GM56350. The U.S. Government has certain rights in this invention.

### BACKGROUND OF THE INVENTION

Certain diseases are characterized by the accumulation of amyloids. For example, amyloid deposits of PrPSc, Aβ, huntingtin, or alpha-synuclein are respectively associated with transmissible spongiform encephalopathies (TSEs), Alzheimer's (AD), Huntington's (HD), and Parkinson's Diseases. Although these proteins differ in primary sequence the amyloid fibrils they form have a similar “cross β” structure. While all proteins can form amyloids because specific side chain reactions are not required, it appears that the ability of the amino acid sequences of most proteins to fold into other stable structure inhibits them from forming amyloids.

Alzheimer's disease (AD) is a severe neurodegenerative disorder characterized by an extracellular deposition of amyloid plaques, and an intraneuronal accumulation of neurofibrillary tangles in the brain of affected individuals. A 42 amino acid long Aβ42 peptide generated by proteolytic processing of the APP protein is a major component of the amyloid plaques, in which it is mainly represented in the form of detergent-insoluble amyloid fibers (reviewed in reference 1). Historically, the Aβ42 fibers have been considered to be the major pathogenic agents of AD. Recently, this hypothesis has been challenged by findings suggesting that fibrillar aggregates may represent inert dead-end products of the Aβ42 aggregation pathway. Considerable evidence now suggests that the primary neurotoxic effects are associated with soluble SDS-stable assemblies of Aβ42, such as 56 kDa Aβ42 dodecamers, or even smaller, low-n (dimers, trimers, and tetramers) oligomers of Aβ42, which seem to appear during the early stages of Aβ42 assembly (reviewed in [1, 3-5]), and could give rise to larger oligomers. Thus, the focus of putative therapeutic interventions has shifted towards unraveling compounds that inhibit the earliest stages of Aβ42 oligomerization. A number of chemical screens have uncovered molecules that inhibit fibrillization of the Aβ42 peptide (reviewed in [6,7]; also see reference 8). These studies, however, did not directly address the issue of inhibiting the earliest stages of Aβ42 assembly, i.e. formation of the SDS-stable soluble low-n oligomers. This aspect is important, as inhibition of the wrong step may lead to accumulation of toxic Aβ42 intermediates.

Fibrils form when denatured or misfolded proteins adapt a β structure which oligomerizes to form SDS stable soluble intermediates culminating in fibril formation. It has been hypothesized that certain normally monomeric proteins tend, upon accumulation, to misfold and form oligomers which exert toxic effects on the neuron. Such proteins evade normal cellular controls of chaperone proteins that help fold proteins properly and selective degradation machinery that rid the cell of misfolded proteins (e.g. the proteasome or lysosome). However, inhibition of fibrillization may lead to the accumulation of toxic oligomers of Aβ42. The formation of oligomers precedes the appearance of fibrils and oligomers are often undetectable once fibrils appear. The model described here can be used to search for and test proteinacious or chemical compounds for their ability to interfere with the initial steps of Aβ42 oligomerization.

Thus, there is a need for a system where one can identify therapeutic agents for diseases associated with protein oligomerization which may have their therapeutic effect due to being either regulators of protein folding, and/or inhibitors of protein aggregation, and/or preventors and/or inhibitors of the process of fibrillogenesis, or those that can have an entirely different and possibly unknown mechanism of action. Furthermore, there is need for a system that will provide a rapid and cost-effective screening method for the identification of agents useful in the treatment, prevention and cure of diseases associated with protein misfolding and/or oligomerization.

Yeast S. cerevisiae is a simple and readily manipulable organism that has been successfully used as a model for various medicinal studies (reviewed in [9,10]), including neurodegenerative disorders, associated with the deposition of amyloid aggregates (see references 11-18). One of the most valuable contributions of yeast biology to the investigation of neurodegenerative disorders in animals was made by studying yeast prions (reviewed in references 19-21). The yeast translational termination factor Sup35p can form self-propagating infectious amyloid aggregates that arise spontaneously in the cell and manifest a prion phenotype referred to as [PSI⁺]. The essential Sup35p protein is composed of three domains. The 124 amino acid long N-terminal domain (N) is glutamine and aparagine rich, dispensable for viability, and required and sufficient for the prion properties of Sup35p. While the function of the highly charged middle (M) domain remains unclear, the C-terminal RF (release factor) domain of Sup35p performs termination of protein translation and is essential for viability.

Prion aggregates of Sup35p are transmitted to daughter cells along with the cytoplasm from the mother cell during cell division [22]. The yeast chaperone Hsp104, a member of the AAA+ protein family [23, 24] is required for the successful maintenance of the [PSI⁺] prion [25]. Hsp104 shears the SDS-stable Sup35p prion amyloid aggregates into smaller structures in an ATP-dependent manner [26, 27] and therefore maintains them in numbers sufficient for the successful transmission to the daughter cell [28, 29]. The ATPase activity of Hsp104 is inhibited by millimolar concentrations of guanidine [30], which is therefore employed as a yeast prion-curing agent [31].

Previous models of Alzheimer's disease have focused on unraveling compounds that inhibit fibrillization of Aβ42, i.e. that last step of Aβ assembly. Fibrils form when denatured or misfolded proteins adapt a β structure which oligomerizes to form SDS stable soluble intermediates culminating in fibril formation. It has been hypothesized that certain normally monomeric proteins tend, upon accumulation, to misfold and form oligomers which exert toxic effects on the neuron. Such proteins evade normal cellular controls of chaperone proteins that help fold proteins properly and selective degradation machinery that rid the cell of misfolded proteins (e.g. the proteasome or lysosome). However, inhibition of fibrillization may lead to the accumulation of toxic oligomers of Aβ42. The formation of oligomers precedes the appearance of fibrils and oligomers are often undetectable once fibrils appear. The model described here can be used to search for and test proteinacious or chemical compounds for their ability to interfere with the initial steps of Aβ42 oligomerization.

A yeast model of the initial steps of Aβ42 oligomerization is described herein. In this model, oligomerization of the Aβ42 peptide was monitored through the activity of the MRF domain of Sup35p, to which the peptide was fused. The model shows that the easily scored activity of Sup35p's MRF domain is impaired in AβMRF fusions because the Aβ42 causes the fusion to form SDS-stable low-n oligomers.

Importantly, only low-n oligomeric species of AβMRF were present in this model, which is in striking contrast to other cell-based models in which such oligomers were never produced. In this model, inhibition of AβMRF oligomerization by specific mutations in the Aβ region restored activity to the fusion. Thus, the system allows the researcher to distinguish between the oligomeric and monomeric forms of AβMRF using a simple test. Using this method, it has been elucidated that guanidine treatment increases, while gene disruption of Hsp104 decreases, oligomerization of the fusion protein. It has been shown that Hsp104 interacts with AβMRF and possibly protects the fusion from degradation and/or disaggregation. The model system allows the researcher to assay oligomerization of AβMRF in the presence as well as in the absence of Hsp104. The latter scenario may be preferred as no mammalian homologue of Hsp104 has been reported. This model system represents a convenient tool to perform chemical and genetic screens for agents that interfere with the earliest steps of Aβ42 oligomerization.

### BRIEF SUMMARY OF THE INVENTION

The present invention is based upon the observation that certain neurodegenerative disorders, associated with the aggregation of amyloid aggregates in parts of the nervous system, are derived in part from the oligomerization of certain peptides and proteins. The herein described screens use the viability of yeast cells, which express peptides or proteins that oligomerize thereby contributing to the formation of toxic intermediates. These peptides and proteins when expressed in yeast serve as the basis for screening for therapeutic agents that interfere with their oligomerization, and thus may be further employed as potential therapeutics against disorders or diseases caused by these oligomers. In addition, proteinacious compounds identified in such screens as regulators of oligomerization of the aformentioned peptides may serve as potential targets for therapeutic intervention. For example, SDS-stable oligomers of a 42 amino acid long Aβ42 peptide are the major contributors to the onset of Alzheimer's disease. The present invention includes methods for screening for therapeutic agents (for example, proteinacious or chemical) that affect Aβ42 oligomerization. The herein described screens use the viability of yeast cells, which express peptides or proteins that oligomerize thereby contributing to the formation of plaques.

In one embodiment, the present invention is a yeast model system focused on the initial stages of Aβ42 oligomerization. This system represents a convenient tool to test or perform chemical and genetic screens for agents that interfere with, for example, the earliest steps of Aβ42 oligomerization. The system centers on a protein fusion between the Aβ42 peptide and the MRF domain of the yeast translation termination factor, Sup35p, and monitoring its activity by the growth of yeast on different media. The presence of the Aβ42 causes the AβMRF fusion protein to form SDS-stable low-n oligomers, which mimics the ability of the natural Aβ42 peptide to form low-n oligomers. The oligomerization of AβMRF compromises its translational termination activity causing a more frequent readthrough of the ade1-14's premature stop codon, or other markers (e.g. ura3-14), which is easily scored by yeast growth (See FIG. 1). Any number of point mutations may be made in any part of the fusion protein. For example, as shown in the below-identified examples, point mutations previously shown to inhibit Aβ42 aggregation in vitro, were made in the Aβ42 portion of the fusion protein. These mutations both inhibited oligomerization and restored activity to the fusion protein.

The herein described system provides a user-friendly assay to determine the degree of AβMRF oligomerization by examining the growth of yeast on complex or selective media. For example, in the below-identified examples, this system has enabled users to demonstrate that Hsp104 regulates the total level of AβMRF and the relative abundance of AβMRF oligomers. It is also shown that the yeast prion curing agent guanidine enhances the level of SDS-stable AβMRF oligomers, presumably by inactivating factors that degrade and/or disaggregate them. This effect was not caused by inactivation of the yeast chaperone Hsp104, which appears to protect AβMRF from the effects of such factors.

The invention also contemplates methods of screening for therapeutic agents for diseases associated with the aggregation of misfolded proteins, for example Alzheimer's disease. These methods may screen for agents that can breakup Aβ42 oligomers, or inhibit their formation. Such methods comprise, for example: (a) contacting one or more yeast cells with a candidate compound, wherein the yeast cells express a fusion protein comprising an amyloid protein or peptide (for example, Aβ42) and one or more domains of Sup35p necessary for translation termination (e.g. MRF) and wherein the yeast cells express one or more marker proteins from alleles having one or more premature stop codons or termination signals (e.g. ade1-14, ura3-14) under conditions that allow for aggregation of the fusion protein, wherein fusion protein aggregation is either essential for yeast cell viability on media lacking the essential nutrient of the molecular pathway in which the marker protein is involved or where fusion protein aggregation causes growth inhibition on media containing an inhibitor of growth (e.g. 5-FOA, if the ura3-14 allele is used) in the presence of the marker protein; (b) measuring the viability of the yeast cell on media lacking the essential nutrient against the viability of the yeast cell on the same media supplemented with the nutrient, or measuring the viability of the yeast cells on media supplemented with growth inhibitor against the viability on media not supplemented with growth inhibitor; (c) comparing the level of viability with the level of viability of a yeast cell not contacted with the candidate compound. There may be a further step if the ade1-14 allele is used: (d) wherein the color of the yeast colonies contacted with the candidate compound on complex medium is assayed and compared with the color of yeast colonies not contacted with the candidate compound.

The present invention further encompasses alternative methods of screening for a therapeutic agent for a protein aggregation disease. These methods may screen for agents which can inhibit the formation of protein aggregates or oligomers, for example Aβ42 oligomers. Such methods comprise, for example: (a) contacting one or more yeast cells with a candidate compound while incubating the yeast cells that express a fusion protein comprising an amyloidogenic protein or peptide (such as Aβ42) and one or more domains of Sup35p necessary for translation termination (e.g. MRF) and wherein the yeast cells express one or more marker proteins from alleles having one or more premature stop codons or termination signals (e.g. ade1-14, ura3-14) under conditions that allow for aggregation of the fusion protein, wherein fusion protein aggregation is either essential for yeast cell viability on media lacking the essential nutrient of the molecular pathway in which the marker protein is involved (e.g. adenine, if ade1-14 is used) or where fusion protein aggregation causes growth inhibition on media containing an inhibitor of growth (e.g. 5-FOA, if the ura3-14 allele is used) in the presence of the marker protein; (b) measuring the viability of the yeast cells on media lacking the essential nutrient against the viability of the yeast cell on the same media supplemented with the nutrient, or measuring the viability of the yeast cells on media supplemented with growth inhibitor against the viability on media not supplemented with growth inhibitor; (c) comparing the level of viability with the level of viability of yeast cells not contacted with the candidate compound. There may be a further step if the ade1-14 allele is used (d) wherein the color of the yeast colonies contacted with the candidate compound on complex medium is assayed and compared with the color of yeast colonies not contacted with the candidate compound.

In some embodiments, the disease associated with the aggregated or oligomerized peptides or proteins is Alzheimer's disease, Parkinson's disease, Familial Amyloid Polyneuropathy, transmissible spongiform encephalopathies (TSEs), Alzheimer's (AD), and Huntington's Disease (HD). The aggregated or oligomerized peptides result in, or co-segregate with, these diseases. It is specifically contemplated that the protein aggregation disease is Alzheimer's disease. In other embodiments the aggregated disease protein is Aβ42, huntingtin, PrP, alpha synuclein, synphilin, transthyretin, tau, ataxin 1, ataxin 3, atrophin, or androgen receptor.

In addition to screening methods, compositions and methods for treatment that arise from the results of screening methods of the invention are also included. In some embodiments of the invention, candidate compounds that are screened may be employed in therapeutic methods and compositions of the present invention. In further embodiments, the candidate compound is determined to be a candidate therapeutic agent based on its performance in screening assays. If cells incubated with the candidate compound cannot grow, or growth is reduced, on medium lacking a nutrient or supplement that is ordinarily derived from the in vivo expression of a gene under the control of the oligomerized or aggregated protein fusion, as compared to cells not incubated with the candidate compound, in some embodiments of the invention the candidate compound is a candidate therapeutic agent. In other embodiments, the candidate compound is a candidate therapeutic agent if cells incubated with the candidate compound grow, or growth is enhanced, on media supplemented with a growth inhibitor; susceptibility to which is ordinarily derived from the in vivo expression of a gene under the control of the oligomerized or aggregated protein fusion, as compared to cells not incubated with the candidate compound. The candidate therapeutic agent may be produced or manufactured, or placed in a pharmaceutically acceptable composition. It is contemplated that any of the screening methods described herein may be employed with respect to therapeutic methods and compositions.

Methods of treating include administering to a patient in need of treatment a therapeutic agent in an amount effective to achieve a therapeutic benefit. A “therapeutic benefit” in the context of the present invention refers to anything that promotes or enhances the well-being of the subject with respect to the medical treatment of his condition, which includes treatment of diseases associated with the abnormal aggregation of proteins, such as Alzheimer's disease. A list of nonexhaustive examples of this includes extension of the subject's life by any period of time, decrease in the number of plaques, fibrils, or oligomers, reduction in fibril growth, reduction in number of protein aggregates, delay in onset of mental capabilities, and a decrease in atrophy, or dementia to the subject that can be attributed to the subject's condition.

In addition to screening methods, and compositions and methods for treatment that arise from the results of screening methods of the invention, the present invention features a variety of yeast strains with highly desirable genetic backgrounds suitable for use in a variety of methods and related kits for practicing the present invention.

As used herein, the term “aggregation” refers to oligomerization and/or a clustering or amassing of two or more peptides or proteins. As used herein, the term “amyloidogenic protein” or “amyloidogenic peptide” refers to any protein or peptide that is a component of amyloid plaques.

### BRIEF DESCRIPTION OF THE DRAWINGS

FIG. 1. AβMRF causes nonsense suppression in yeast. (Upper panel) Schematic illustration of the constructs used in this study: (a) full length Sup35p (NMRF) (b) Sup35p without the N-terminal prion domain (MRF) (c) Aβ42 fused to the N terminus of MRF (AβMRF) (d) AβMRF carrying a double mutation of Phe19,20Thr in its Aβ42 portion (Aβm1MRF) (e) AβMRF carrying a triple mutation of Phe19,20Thr and Ile31Pro in its Aβ42 portion (Aβm2MRF). All these constructs carry an HA tag between the M and RF domains. Images shown are not to scale. (Lower panel) Equal numbers of ade1-14 cells containing a genomic deletion of SUP35 (sup35Δ), and carrying the indicated constructs (a-e) on a plasmid were grown on complex medium, or synthetic medium supplemented (+Ade) or not (−Ade) with adenine. (a) Cells with inactivated NMRF ([PSI+]) had an impaired translational termination activity, were white and grew on −Ade. (b) Cells with fully active MRF (lacking the aggregation-prone prion, N, domain), were red and failed to grow on −Ade. (c) Cells expressing AβMRF have an impaired translational termination activity, as they were white and grew on −Ade. (d, e) The translational termination activity was restored by F19,20T (Aβm1MRF) and F19,20T/131P (Aβm2MRF) mutations in the Aβ42 region of the fusion protein, making the cells dark pink and preventing their growth on −Ade.

FIG. 2. AβMRF forms SDS-stable oligomers in yeast. (A) Immunoblot analysis of lysates from sup35Δ cells containing prionized NMRF ([PSI+]) or other indicated constructs. Lysates were treated with 1% SDS for 7 mins at room temperature and resolved by electrophoresis in agarose. Immunoblot analysis was performed using anti-RF antibodies, followed by stripping and staining with anti-Aβ antibodies. The positions of molecular weight standards, treated identically to the experimental samples, are shown (calc., calculated position). AβMRF formed SDS-stable low-n oligomers that largely disappeared after the introduction of the F19,20T (Aβm1MRF) and F19,20T/131P (Aβm2MRF) mutations into the Aβ42 portion of the fusion protein. The decreased efficacy with which anti-Aβ antibodies recognized oligomers of AβMRF suggests that oligomerization occurred through the Aβ42 portion of the fusion protein. (B) 5 mg of amyloid fibers of Aβ42 peptide were treated with 1% SDS, resolved in agarose and analyzed by immunoblotting with anti-Aβ antibodies. Only a fraction of Aβ42 fibres can enter the 1.5% agarose gel. (C) Same as in (A) but the samples were resolved in an acrylamide gel. Asterisk denotes non-specific antibody interaction.

FIG. 3. Guanidine stimulates oligomerization of AβMRF. sup35Δ cells expressing the indicated constructs were grown in the absence (−) or presence (+) of 6.3 mM guanidine (Gu). Equal amounts of lysate proteins were treated with 1% SDS and analyzed by immunoblotting with anti-RF or anti-Aβ antibodies following electrophoresis in agarose. Equal protein loading on each panel was confirmed by coomassie staining of the membrane (not shown).

FIG. 4. Deletion of HSP104 decreases the total amount of AβMRF and reduces the proportion of oligomers. (A) AβMRF or Aβm2MRF were expressed in a sup35Δ strain in the presence (WT) or absence (Δ) of HSP104. Equal amounts of lysate proteins were treated with 1% SDS and analyzed by immunoblotting with anti-RF antibodies following electrophoresis in agarose. Equal protein loading was confirmed by coomassie staining of the membrane (not shown). (B) The effects of HSP104 deletion on the total amount of AβMRF and the ratio between oligomers and monomers from panel A were evaluated by densitometry. The height of the bars reflects total amount of AβMRF relative to that in HSP104 WT cells (error bars: s.e., n=3). Each bar is subdivided according to the content of oligomers (open) and monomers (shaded) of AβMRF (±s.e., n=3). The deletion of HSP104 decreased the total amount of AβMRF and decreased the ratio of oligomers to monomers.

FIG. 5. Deletion of HSP104 exacerbates the translation termination defect of AβMRF. Equal numbers of sup35Δ yeast containing (WT) or lacking (Δ) HSP104 and expressing AβMRF or Aβm2MRF were grown on complex medium, or synthetic medium supplemented (+Ade) or not (−Ade) with adenine. Deletion of HSP104 stimulated growth of AβMRF-expressing cells on −Ade, while having no effect on yeast grown on +Ade medium.

FIG. 6. Guanidine stimulates oligomerization of AβMRF in the absence of HSP104. AβMRF-expressing sup35Δ hsp104 Δcells were grown in the absence (−) or presence (+) of 6.3 mM guanidine (Gu). Equal amounts of lysate proteins were treated with 1% SDS and analyzed by immunoblotting with anti-RF antibodies following electrophoresis in agarose. Equal protein loading was confirmed by coomassie staining of the membrane (not shown).

FIG. 7. Co-immunoprecipitation of Hsp104 with AβMRF. Lysates of sup35Δ cells with (WT) or without (Δ) HSP104, expressing non-tagged NMRF, HA-tagged MRF, or HA-tagged AβMRF, were incubated with anti-HA antibodies immobilized on agarose beads. Co-precipitated proteins were eluted and analyzed by immunoblotting with anti-RF and anti-Hsp104 antibodies. Hsp104 co-immunoprecipitated with AβMRF, but not with MRF. Non-HA-tagged NMRF was used as a control for non-specific binding to anti-HA antibodies.

FIG. 8. Positive selection for translational readthrough. A [psi−] and [PSI+] version of a strain bearing the ade1-14 and newly constructed ura3-14 markers with premature stop codons was spotted on the indicated medium. The [psi−] cells fail to readthrough the premature stop codon mutations so the cells are red (because of ade1-14) and unable to grow on −Ura but able to grow on +FOA (because of ura3-14). Cells with the [PSI+] prion, which cause readthrough, are white, Ura3+, and unable to grow on +FOA. Thus, the level of growth on +FOA provides a measure of the efficiency of translational readthrough in ura3-14 cells.

### DETAILED DESCRIPTION OF THE INVENTION

The present invention is directed to novel compositions and methods for screening agents that interfere with Aβ42 oligomerization. In some embodiments these agents prevent oligomerization. Irrespective of the exact mechanism of action, agents identified by the screening methods of the invention will provide therapeutic benefit to diseases involving protein aggregation, oligomerization, misfolding or aberrant protein deposition. Non-limiting examples of such diseases include: Alzheimer's disease, Huntington's disease, and Parkinson's disease.

The invention also contemplates methods of screening for therapeutic agents for diseases associated with the aggregation of misfolded proteins, for example Alzheimer's disease. These methods may screen for agents that can breakup Aβ42 oligomers, or inhibit their formation. Such methods comprise, for example, (a) contacting one or more yeast cells with a candidate compound, wherein the yeast cells express a fusion protein comprising Aβ42 and one or more domains of Sup35p necessary for translation termination and wherein the yeast cells express one or more marker proteins from alleles having one or more premature termination signals, under conditions that allow for aggregation of the fusion protein, wherein fusion protein aggregation is essential for yeast cell viability on media lacking the essential nutrient of the molecular pathway in which the marker protein is involved; (b) measuring the viability of the yeast cell on media lacking the essential nutrient against the viability of the yeast cell on the same media supplemented with the nutrient; (c) comparing the level of viability with the level of viability of a yeast cell not contacted with the candidate compound. This method may further comprise a step (d) wherein the color of the yeast colonies contacted with the candidate compound on complex medium is assayed and compared with the color of yeast colonies not contacted with the candidate compound. This step (d) is particularly useful when the marker protein is Ade1p expressed from an ade1-14 allele.

In another embodiment, alternative methods of screening for compounds that decrease aggregation of amyloidogenic proteins are described. These methods comprise, for example, (a) contacting a yeast cell with a candidate compound wherein the yeast cell expresses a fusion protein comprising an amyloidogenic peptide or protein (such as Aβ42) and one or more domains of Sup35p necessary for translation termination and wherein the yeast cell expresses one or more marker proteins having one or more premature termination signals, under conditions that allow for aggregation of the fusion protein, wherein fusion protein aggregation causes growth inhibition on media containing an inhibitor of growth in the presence of the marker protein; (b) measuring the viability of the yeast cell on media not supplemented with growth inhibitor; (c) comparing the level of viability with the level of viability of a yeast cell not contacted with the candidate compound. An example of a growth inhibitor is 5-fluoroorotic acid (5-FOA). An example of a marker protein that can be used in the foregoing method is Ura3p expressed from a ura3-14 allele.

As described herein, the screening methods of the invention use yeast cells that are engineered to express one or more marker genes having one or more nonsense mutations. Marker genes and proteins are well known in the art. The marker genes may be, for example, selected from ADE1, LYS2, LYS5, CAN1, MET2, MET15, GAL1 and URA3. In addition, the yeast cells contain one or more mutations in their genomic SUP35 gene (encoding a yeast translation termination factor) such that the expressed protein is non-functional. Alternatively, the yeast strain may harbor a genomic deletion of the SUP35 gene; for example, a sup354::LEU2 disruption. In another embodiment, the yeast cells may contain a double knockout, or deletion, of SUP35 and HSP104 (for example, sup35Δ::LEU2 and hspΔ::URA3). The herein described screens identify candidate compounds that decrease translational readthrough at the one or more nonsense mutation(s) introduced into the one or more marker genes. Yeast marker genes are well known in the art. Two such genes are ADE1 and URA3. Translational readthrough of the ade1-14 nonsense mutation causes ade1-14 cells, which are red on complex medium and unable to grow on −Ade medium, to become lighter in color on complex medium and to grow on −Ade medium. The herein described screens identify candidate compounds that decrease translational readthrough caused by Aβ42-M-RF oligomerization (wherein the M and RF domains of the Aβ42-M-RF fusion correspond to the middle and release factor domains of Sup35p).

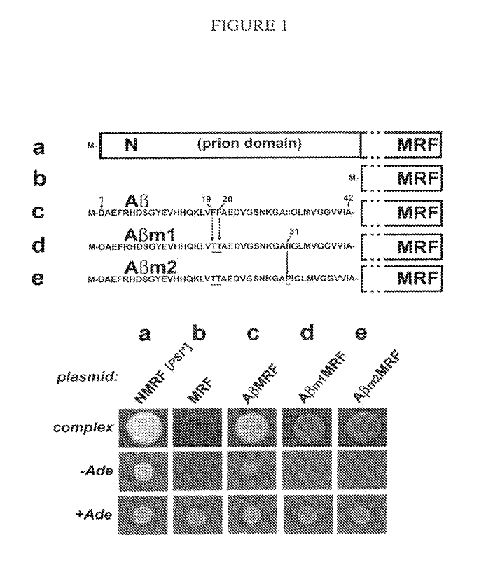

The activity of the essential translational termination factor Sup35p (NMRF) is conveniently assayed in vivo by examining the efficiency with which protein synthesis terminates at a premature stop codon (a nonsense-suppression assay, for review see [2, 33]; FIG. 1). The assay may use the ade1-14 nonsense allele. Strains carrying this mutation and bearing fully active NMRF produce only a truncated (inactive) version of Ade1p, and as a result cannot grow on synthetic medium lacking adenine (−Ade), while they grow normally on synthetic medium supplemented with adenine (+Ade). In addition, these cells accumulate a red intermediate of the adenine synthesis pathway when grown on complex medium. However, if the efficiency of translational termination at the premature stop codon of the ade1-14 allele is compromised, the cells gain the ability to grow on −Ade (i.e. they become Ade⁺) and do not accumulate red pigment. For example, cells expressing the complete Sup35p containing the N (prion), M (middle), and RF (release factor) domain, are white and Ade⁺ when NMRF is in the aggregated [PSI⁺] prion form (FIG. 1a). Cells expressing an aggregation-deficient and therefore fully functional form of Sup35p lacking the non-essential N-terminal domain (MRF) are red and Ade⁻ (FIG. 1b). Thus, this well established system reliably distinguishes between fully active monomer, and malfunctioning aggregated forms of NMRF [19, 25, 34].

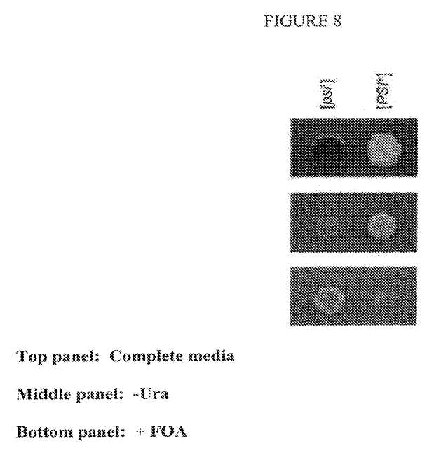

The ura3-14 allele of the URA3 gene is another useful marker that can be used in the presently described methods. The ura3-14 allele has a premature stop codon and is useful because one can positively select for inactivation of Ura3. Cell producing Ura3p cannot, while cells lacking Ura3p can, grow on medium containing 5-fluoroorotic acid (+5-FOA). Without the translational readthrough ura3-14 cells do not produce Ura3p and therefore grow on medium containing 5-FOA. With the translational readthrough ura3-14 cells produce Ura3p and therefore cannot grow on medium containing 5-FOA. Using this allele, one can select for drugs that reduce translational readthrough caused by the Aβ42-M-RF oligomerization by selecting for increased growth on +FOA medium. See FIG. 8, for example.

Any yeast strain may be used in context of the present invention. Some examples of yeast cell strains that can be used in the present method include Saccharomyces uvae, Saccharomyces kluyveri, Schizosaccharomyces pombe, Saccharomyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp. and Geotrichum fermentans. The preferred yeast strain is Saccharomyces cerevisiae.

As the invention concerns screening methods of a wide-variety of pharmaceutical, chemical and genetic agents, one concern is that some of the candidate substances may not be either permeable into yeast cells, or may not be taken up by yeast cells, or may be rapidly metabolized once they enter into the yeast cell, or may be pumped out of the yeast cell. The present inventors contemplate using suitable mutations of yeast strains designed to eliminate these problems. In one example, a yeast strain bearing mutations in 3 genes, the ERG6, PDR1, and PDR3, which affect membrane efflux pumps and increasing permeability for drugs are contemplated of use. This particular strain has been used successfully in cancer research to identify growth regulators.

Methods of using the herein described yeast strains and kits having one or more of these strains as components thereof are also contemplated by the present invention. The strains of the present invention may also be included as one of many components in a kit. For example, a kit may include a yeast strain containing a genomic deletion of SUP35 (sup35Δ::LEU2), or a double deletion of SUP35 and HSP104 (hsp104Δ::URA3), either of which harboring, for example, an AβMRF fusion under the control of an inducible promoter. The fusion protein may alternatively comprise any amyloidogenic protein and one or more domains of Sup35p. The strain may have a genomic background such as: MATa ade1-14 ura3-52 leu2-3,112 trp1-289 his3-200, wherein the ade1-14 allele represents a marker for which AβMRF aggregation can be easily measured. These kits are useful in for screening compounds that inhibit protein aggregation.

A “candidate compound” or “candidate drug” or “test compound” or “test drug” as used herein, is any substance with a potential to reduce, alleviate, prevent, or reverse the oligomerization or aggregation of Aβ42. Various types of candidate compounds may be screened by the methods of the present invention. Genetic agents can be screened by contacting the yeast cell with a nucleic acid construct encoding a gene which encodes a protein that can be expressed in the yeast cell. For example, one may screen cDNA libraries expressing a variety of proteins to identify therapeutic genes or proteins for the diseases described herein. In other examples, one may contact the yeast cell with other proteins or polypeptides which may confer the therapeutic effect.

Thus, candidate substances that may be screened according to the methods of the invention include those encoding chaperone molecules, heat shock proteins, receptors, enzymes, ligands, regulatory factors, and structural proteins. Candidate substances also include nuclear proteins, cytoplasmic proteins, mitochondrial proteins, secreted proteins, plasmalemma-associated proteins, serum proteins, viral antigens, bacterial antigens, protozoal antigens and parasitic antigens. Candidate substances additionally comprise proteins, lipoproteins, glycoproteins, phosphoproteins and nucleic acids (for example, RNAs such as ribozymes or antisense nucleic acids). Proteins or polypeptides which can be screened using the methods of the present invention include chaperone proteins, hormones, growth factors, neurotransmitters, enzymes, clotting factors, apolipoproteins, receptors, drugs, oncogenes, tumor antigens, tumor suppressors, structural proteins, viral antigens, parasitic antigens and bacterial antigens. In addition, numerous methods are currently used for random and/or directed synthesis of peptide, and nucleic acid based compounds. The nucleic acid or protein sequences include the delivery of DNA expression constructs that encode them.

In addition, candidate substances can be screened from large libraries of synthetic or natural compounds. One example, is a FDA approved library of compounds that can be used by humans. In addition, synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.) and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (N.C.), or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors. Additionally, natural and synthetically produced libraries and compounds are readily modified through conventional chemical, physical, and biochemical means.

Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Useful compounds may be found within numerous chemical classes, though typically they are organic compounds, including small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, triterpenoid compounds, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

### Example 1

### Model of Aβ42 Oligomerization

The activity of the essential translational termination factor Sup35p (NMRF) is conveniently assayed in vivo by examining the efficiency with which protein synthesis terminates at a premature stop codon (a nonsense-suppression assay, for review see [2, 33]; FIG. 1). The assay uses the ade1-14 nonsense allele. Strains carrying this mutation and bearing fully active NMRF produce only a truncated (inactive) version of Ade1p, and as a result cannot grow on synthetic medium lacking adenine (−Ade), while they grow normally on synthetic medium supplemented with adenine (+Ade). In addition, these cells accumulate a red intermediate of the adenine synthesis pathway when grown on complex medium. However, if the efficiency of translational termination at the premature stop codon of the ade1-14 allele is compromised, the cells gain the ability to grow on −Ade (i.e. they become Ade⁺) and do not accumulate red pigment. For example, cells expressing the complete Sup35p containing the N (prion), M (middle), and RF (release factor) domain, are white and Ade⁺ when NMRF is in the aggregated [PSI⁺] prion form (FIG. 1a). Cells expressing an aggregation-deficient and therefore fully functional form of Sup35p lacking the non-essential N-terminal domain (MRF) are red and Ade⁻ (FIG. 1b). Thus, this well established system reliably distinguishes between fully active monomer, and malfunctioning aggregated forms of NMRF [19, 25, 34].

To establish a model of Aβ42 oligomerization in S. cerevisiae, we fused the Aβ42 peptide with MRF (Sup35p lacking the N-terminal domain), and containing an HA tag between the M and RF domains. The resulting protein, AβMRF (see upper panel of FIG. 1 for constructs used in this study) was mutagenized in its Aβ42 portion according to a recent model of Aβ42 oligomerization [35]. The model suggests that binding of one Aβ42 molecule to another occurs through four regions: amino acids 15-21, 24-32, 35-37, and 40-42 of one molecule bind to the corresponding regions in another molecule. Substitutions of Phe19, Phe20, and Ile31 were previously shown to inhibit aggregation of Aβ42 in vitro and prevent its neurotoxic effects [35-37]. To obtain an oligomerization-deficient control for the AβMRF fusion protein, we disrupted the first and the second aggregation-important regions of Aβ42 by making double Aβ^{F19,20T}MRF (Aβm1MRF) or triple Aβ^{F19,20T/I31P}MRF (Aβm2MRF) substitutions in the Aβ42 portion of AβMRF. These constructs were expressed in a 74D-694 (ade1-14) sup35Δ strain, and therefore were the only sources of the essential Sup35p's RF domain.

Yeast cells expressing the AβMRF fusion protein were white on complex medium and grew on −Ade, suggesting that the translation termination activity of the fusion protein was impaired (FIG. 1). In contrast, yeast expressing Aβm1MRF or Aβm2MRF were dark pink on complex medium and Ade⁻, suggesting that the efficiency of translation termination was almost completely restored by the mutations in the Aβ42 portion of the fusion protein. No growth difference was detected in the control experiment on +Ade medium. These results are consistent with the hypothesis that the presence of Aβ42 in the fusion protein caused it to aggregate into SDS-stable oligomers, thereby affecting its translation termination activity.

### Example 2

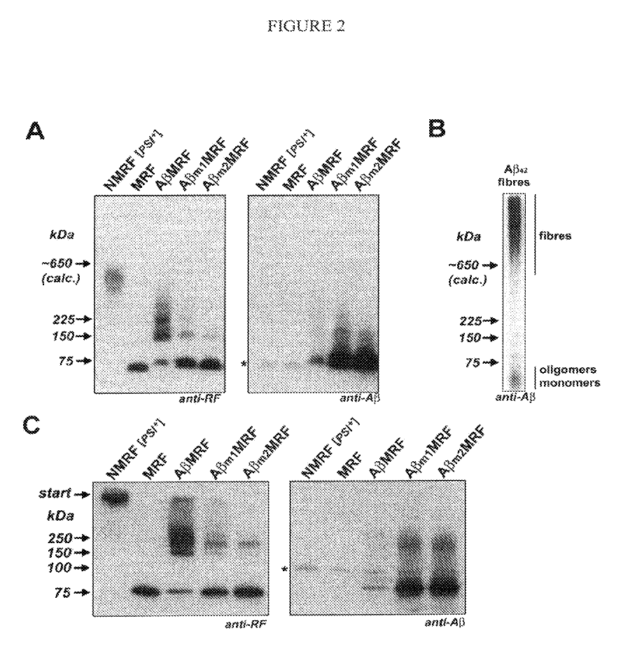

To test whether AβMRF formed SDS-stable oligomers, we analyzed yeast lysates treated with 1% SDS at room temperature by immunoblotting. As shown elsewhere [26], prionized NMRF ([PSI₊]) migrates in the form of SDS-stable aggregates, while MRF, which is unable to prionize, is monomeric (FIG. 2). The pool of AβMRF contained both monomers and SDS-stable complexes migrating at the predicted positions for AβMRF low-n oligomers (dimers, trimers, and tetramers) (FIG. 2). In agarose gels, the AβMRF monomers (calculated molecular weight ˜73.7 kDa) migrated at ˜65 kDa (FIG. 2A), rather than at ˜77 kDa as they did in the acrylamide gels (FIG. 2C). Nevertheless, the positions of the SDS-stable complexes increased with monomer size increments in both gel systems. The SDS-stable oligomers of AβMRF were able to withstand treatment with 2% SDS at room temperature and disaggregated into monomers only after boiling (not shown). We hypothesize that the presence of Aβ42 confers AβMRF with the ability to form low-n oligomers (dimers, trimers, and tetramers) similar to the oligomerization of the Aβ42 peptide in vitro and in the human brain [38-41].

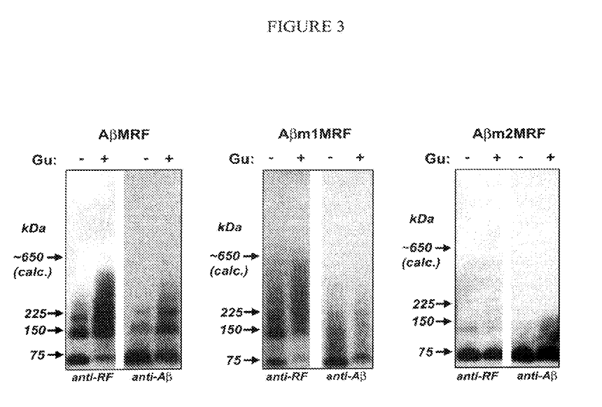

Unlike antibodies against the RF domain, the efficacy with which anti-Aβ antibodies recognized oligomers of AβMRF decreased as the size of the oligomers increased (FIG. 2A,C, compare left and right panels). Nevertheless, oligomers of AβMRF were stably detected by these antibodies (FIG. 3, left panel). We observed a similar phenomenon when we detected SDS-stable amyloid oligomers of NMRF, which represent structures interacting through the ˜14 kDa N-terminal domains, while the ˜70 kDa MRF domains on their C termini are exposed to the solvent. The efficiency with which these oligomers are detected with anti-N terminal antibodies is lower that with antibodies against the C-terminal RF domain [42, 43]. Apparently, even after SDS electrophoresis the physical access of the antibodies to the target epitopes remains impeded due to the assembly state of the target protein. These observations further corroborate our hypothesis that AβMRF molecules are oligomerized through their Aβ42 portions.

### Example 3

To obtain additional evidence that it is the intact Aβ42 peptide fused to the MRF that conferred the AβMRF fusion protein with the ability to form low-n oligomers, we analyzed point mutants of AβMRF by SDS-electrophoresis and immunoblotting. We expected that mutations in the aggregation-important regions of Aβ42 would inhibit oligomerization of the fusion protein. Consistent with our expectations, disruption of a single aggregation-important region of Aβ42 (Aβm1MRF) reduced its ability to form low-n oligomers (FIG. 2). Disruption of a second aggregation-important region (Aβm2MRF) further inhibited oligomerization of the protein. However, small amounts of Aβ m2MRF were found in the form of dimers. This is probably due to the fact that this construct still retained two out of four aggregation-important regions intact. The presence of Aβm2MRF dimers explains our observation that these cells were dark pink on complex medium, and were not entirely as red as when a non-tagged MRF protein was expressed (FIG. 2). In addition, the mere presence of Aβ42 on the N-terminus of MRF might slightly inhibit the translation termination activity of the fusion protein. Nevertheless, the fact that point mutations in the aggregation-important regions of Aβ42 impeded oligomerization of AβMRF and restored its activity, illustrates the ability of the system to clearly distinguish between different levels of AβMRF oligomerization.

We did not detect AβMRF oligomers using the generic oligomer-specific antibodies that recognize oligomers of different amyloidogenic proteins ([44], and data not shown). These antibodies appear not to recognize oligomers of Aβ42 smaller than octamers [44], and the oligomers of AβMRF did not reach this size. Nor could we detect AβMRF-containing structures that correspond to fibres of AβMRF, although we successfully used SDS-electrophoresis in agarose previously to analyze different amyloid fibres [43]. In FIG. 2B we show that amyloid fibres made of recombinant Aβ42 peptide are detected in the upper part of the agarose gel, while AβMRF never formed structures of this size (FIG. 2A). It is possible that the absence of large AβMRF assemblies can be attributed to disaggregating activity of unknown cellular factors, or that the presence of a large MRF domain impedes the ability of AβMRF to assemble into structures larger than the low-n oligomers.

### Example 4

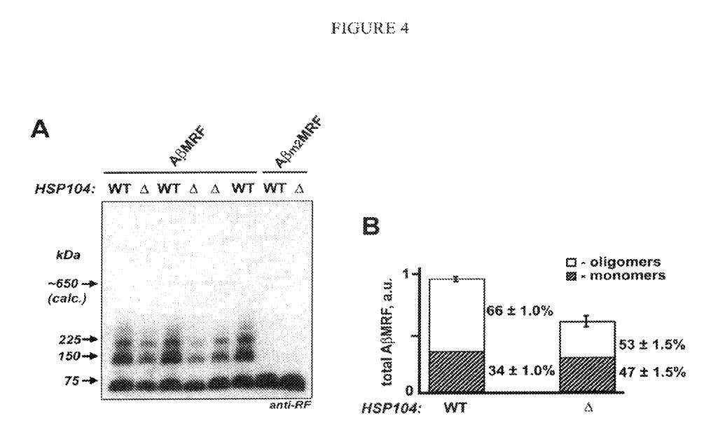

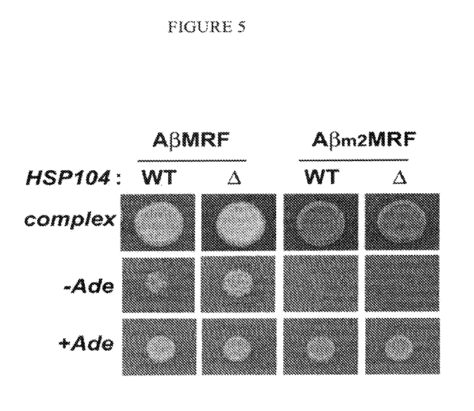

As growth of [PSI⁺] yeast in the presence of the prion curing agent guanidine [31] increased the size of SDS-resistant Sup35p aggregates [26], we wondered if it would affect AβMRF oligomers similarly. Thus we grew sup35Δ cells expressing AβMRF, Aβm1MRF, or Aβm2MRF in the presence of 6.3 mM guanidine. Strikingly, guanidine dramatically increased the amount of AβMRF and Aβm1MRF oligomers, while depleting the monomeric pool of the proteins (FIG. 3). No such effect was detected with the control Aβm2MRF protein. As guanidine enhances the Sup35p [PSI⁺] aggregate size by inhibiting the ATPase activity of Hsp104, we wondered if guanidine's effect on the accumulation of AβMRF oligomers was likewise due to Hsp104 inhibition. Contrary to this hypothesis, deletion of HSP104 decreased the proportion of AβMRF oligomers in three independent hsp104Δ clones (FIG. 4). In addition, hsp104Δ led to a decrease in the total amount of AβMRF in the cells by ˜40%, while having no effect on the level of Aβm2MRF protein (FIG. 4). As might be expected, such a decrease in the total amount of AβMRF in hsp104Δ cells resulted in more frequent readthrough of the premature stop codon of the ade1-14 allele: hsp104Δ caused AβMRF-expressing sup35Δ cells to grow slightly better on −Ade, while having no effect on Aβm2MRF-expressing sup35Δ cells (FIG. 5).

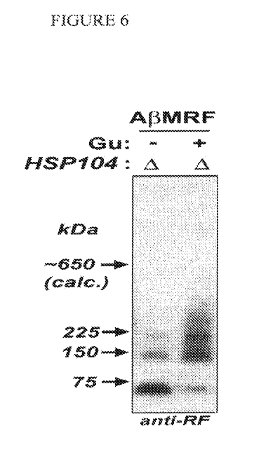

These results suggest that Hsp104 is not the target of guanidine that stimulated AβMRF oligomerization, and that guanidine therefore may affect other cellular factors. Corroboratively, guanidine stimulated oligomerization of AβMRF even in the absence of HSP104 (FIG. 6).

### Example 5

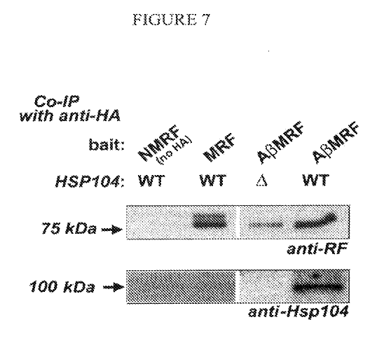

To test if Hsp104 interacts with AβMRF, we used HA-tagged AβMRF and HA-tagged MRF to co-immunoprecipitate Hsp104. Indeed, Hsp104 co-immunoprecipitated with AβMRF, but not with MRF (FIG. 7). This is consistent with observations made elsewhere [45] suggesting that Hsp104 interacts with Aβ42 in vitro. We hypothesize that in our system oligomers of AβMRF undergo continuous disaggregation, and at the same time oligomers and monomers of AβMRF undergo degradation, as a result of interaction between an unknown cellular factor(s) and the Aβ42 portion of AβMRF. We show that Hsp104 binds to the Aβ42 portion of AβMRF and may therefore physically impede interaction between Aβ42 and the factors that trigger degradation and disaggregation of the fusion protein. Consistent with this, deletion of HSP104 led to a ˜40% decrease in the total amount of the AβMRF protein (FIG. 4), possibly as a result of increased susceptibility of AβMRF to degradation-triggering factors. At the same time, deletion of HSP104 shifted the equilibrium between oligomers and monomers such that the monomer's share in the overall pool of AβMRF increased from 34 to 47%, possibly as a result of AβMRF disaggregation. As disaggregation of protein aggregates in yeast usually requires energy from ATP [46-48], it is tempting to speculate that guanidine may specifically inhibit the ATPase activity of the unknown disaggregating factors, as guanidine is able to inhibit the ATPase activity of Hsp104 [30].

Recent evidence suggests that chaperones play critical roles in protecting neuronal cells from the deleterious effects of amyloid aggregates and their precursors (reviewed in [49]). Such a protective mechanism may involve degradation and/or disaggregation of toxic intermediates. In yeast, disaggregation of aggregated protein is carried out by the chaperone machinery, which includes Hsp104, Hsp70/Hsp40, and small heat shock proteins (sHsp) Hsp42, and Hsp26 [23, 46, 50, 51]. All of these chaperones except for Hsp104 have homologs in mammals. It was shown that Hsp26 facilitates disaggregation and refolding of thermally denatured firefly luciferase [47] and citrate synthase [48] by Hsp104 and Hsp70/Hsp40. The disaggregating activity of the yeast chaperone machinery is not limited to amorphous protein aggregates. Overexpression of Hsp104 together with Hsp26 and Hsp42 [47], or Hsp70 together with Hsp40 [52], or Hsp70 alone [18] increased the solubility of polyglutamine aggregates in yeast models of Huntington's disease, while deletion of HSP104 led to solubulization of polyglutamine aggregates [53]. The direct implication of chaperone machinery to the pathology of Alzheimer's disease is still obscure. The yeast system described in this study provides an opportunity to examine the ability of different compounds (proteinacious or chemical) to interfere with the process of AβMRF oligomerization. Abrogation of oligomerization of the AβMRF will lead in our system to the accumulation of AβMRF monomers, causing inhibition of growth in the absence of adenine, and a redder color on complex medium, thus providing a simple functional readout.

### Example 6

### A Suppressible Allele

Translational readthrough of the ade1-14 nonsense mutation causes ade1-14 cells which are red and unable to grown on −Ade medium to become lighter in color and to grow on −Ade. Our screen fro drugs that decrease translational readthrough caused by the Aβ42-M-RF oligomerization can be achieved using ade1-14, by looking for cells that become redder or that have reduced growth on −Ade. Since many drugs may reduce growth rate in general any screen involving reduced growth on −Ade would have to include controls showing that the drug does not cause reduced growth on +Ade medium. It may be easier to use an assay in which drugs that reduce readthrough cause cells to have an increase in growth rate. One such marker is URA3 since there is a positive selection for inactivation of Ura3p because ura3 mutant cells, but not Ura+ cells, can grow on medium containing 5-fluoroorotic acid (+5FOA).

A suppressible allele which contains a nonsense mutation in the URA3 gene was constructed. This allele can be used to select for drugs that reduce translational readthrough caused by the Aβ42-M-RF oligomerization by selecting for increased growth on +FOA medium. See FIG. 8.

### Example 7

### Methods

### Yeast Strains and Media

Derivatives of yeast strain 74D-694 (MATa ade1-14 ura3-52 leu2-3,112 trp1-289 his3-200 [54]) containing a genomic deletion of SUP35 (sup35Δ::LEU2), or a double deletion of SUP35 and HSP104 (hsp104Δ::URA3) [55] (kind gifts from Drs. C. G. Crist and Y. Nakamura) were used in this study. Since the RF domain of Sup35p is essential, viability of the sup35Δ 74D-694 (L2723) and hsp104Δ sup35Δ 74D-694 (L2725) strains was maintained by a pRS313-based (CEN, HIS3) plasmid encoding full length Sup35p [56]. A [PSI⁺] derivative of the sup35Δ 74D-694 strain was described earlier [56]. For this study, plasmids encoding full length Sup35p in L2725 and L2723 and were replaced with pRS313 or pRS316-based (CEN, URA3) plasmids encoding MRF, AβMRF, or aggregation-deficient derivatives of AβMRF (see below). The absence of the original full length Sup35p was confirmed by immunoblotting with polyclonal antibodies against Sup35p's N domain (Ab0332, a kind gift from Dr. S. Lindquist).

Standard yeast media, cultivation and transformation procedures were used [57]. Yeast was cultivated either in complex medium (YPD: 2% dextrose, 2% bacto peptone, 1% yeast extract), or in complete synthetic medium (an artificial mix of 2% dextrose and all necessary aminoacids and nucleobases) lacking adenine (−Ade), uracil (−Ura), or histidine (−His), as required. Complete synthetic medium was referred in the text as ‘+Ade’ medium. As the RF domain within the fusion proteins is essential, no plasmid selection was required after the strains acquired the desired RF-containing constructs. Expression of the AβMRF constructs driven by the copper-inducible CUP1 promoter was stimulated by the addition of 50 μM CuSO₄ to all media. Where indicated, the media were supplemented with 6.3 mM guanidine hydrochloride. 5-FOA media is a complete synthetic media containing 1.7 g/l Yeast Nitrogen Base, 5 g/l ammonium sulphate, 1 g/l 5-FOA.

### Plasmid Construction

The pRS316-based CEN URA3 plasmid (p1071) encoding full length Sup35p under its native promoter with an HA tag between the M and RF domains, and with the NM domains surrounded by BamHI sites was kindly supplied by Dr. J. Weissman [58]. To construct MRF (HA-tagged Sup35p without the 123 N-terminal amino acids which constitute the prion, N, domain of Sup35p) under its native promoter, a fragment containing the HA-tagged M domain (M^{HA}) and a new BamHI site (introduced on primer 1) was PCR amplified from p1071 using primers 1 and 2. The PCR product was cut with BamHI and inserted into p1071 cut with the same enzyme, resulting in p1366, where M replaced NM.

To construct AβMRF under the copper-inducible CUP1 promoter (p1364), we PCR amplified a DNA fragment encoding Aβ42 flanked by restriction sites, using the overlapping primers 3, and 4, which we designed based on the known amino acid sequence of the peptide (DAEFRHDSGYEVHHQKLVFFAEDVGSNKGAIIGLMVGGVVIA) (SEQ ID NO:11). The resulting PCR product was cut with BamHI and BglII and inserted in the correct orientation into p1071 cut with BamHI, yielding p1300, where Aβ42 replaced NM. The native SUP35 promoter in p1300 was replaced with the CUP1 promoter from p984 [59] using XhoI and BamHI sites, yielding p1301. A fragment containing M^{HA} and a new Eco521 site (introduced on primer 5) was PCR amplified from p1071 using primers 5 and 6, cut with Eco521 and inserted into p1301 cut with the same enzyme in the linker region between Aβ42 and RF, resulting in the following construct: CUP1::met-Aβ42-HA-M-3×HA-RF (p1364) referred to herein as AβMRF.

A double substitution in the Aβ42 region of AβMRF (Aβ42^{F19,20T}MRF or Aβm1MRF) was introduced into p1364 by site-directed mutagenesis using a Quick-Change (Stratagene) kit, as suggested by the manufacturer, using primers 7 and 8, resulting in p1397. This plasmid was further mutagenized using primers 9 and 10, to obtain Aβ42^{F19,20T/I31P}MRF, or Aβm2MRF (p1541).

To shuffle the AβMRF fusions into pRS313 (CEN, HIS3), corresponding fragments encoding the fusion proteins with their CUP1 promoters were cut from p1364, p1397, and p1541 with Sad and XhoI and inserted into pRS313 cut with the same enzymes, yielding p1547, p1549 and p1551, respectively.

The expression level and the oligomeric pattern of all corresponding AβMRF fusions expressed from the sibling shuffle vectors pRS313 (CEN, HIS3) and pRS316 (CEN, URA3) were the same (not shown).

List of primers (5′-3′):

(SEQ ID NO: 1)

| | |
|---|---|
| 1. | GGTTTCCAAGGATCCTCTCAAGGTATGTC |

(SEQ ID NO: 2)

| | |
|---|---|
| 2. | CCACCAAACATCCATGGGAATTCTGC |

(SEQ ID NO: 3)

| | |
|---|---|
| 3. | AGCTGGATCCATGGATGCAGAATTCCGACATGACTCAGGATATGA |
| | AGTTCATCATCAAAAATTGGTGTTCTTTGCAGAAGATGTG |

(SEQ ID NO: 4)

| | |
|---|---|
| 4. | ATTAAGATCTCGCTATGACAACACCGCCCACCATGAGTCCAATGA |
| | TTGCACCTTTGTTTGAACCCACATCTTCTGCAAAGAACAC |

(SEQ ID NO: 5)

| | |
|---|---|
| 5. | TCTCACGGCCGGTCTTTGAACGACTTTC |

(SEQ ID NO: 6)

| | |
|---|---|
| 6. | CCACCAAACATCCATGGGAATTCTGC |

(SEQ ID NO: 7)

| | |
|---|---|
| 7. | CATCATCAAAAATTGGTGACCACTGCAGAAGATGTGG |

(SEQ ID NO: 8)

| | |
|---|---|
| 8. | CCACATCTTCTGCAGTGGTCACCAATTTTTGATGATG |

(SEQ ID NO: 9)

| | |
|---|---|
| 9. | GGGTTCAAACAAAGGTGCACCAATTGGACTCATGGTGGGCGG |

(SEQ ID NO: 10)

| | |
|---|---|
| 10. | CCGCCCACCATGAGTCCAATTGGTGCACCTTTGTTTGAACCC |

All plasmids used in this study were analyzed by restriction analysis and sequencing, and their protein products were tested by immunoblot analysis with antibodies against Aβ (6E10), RF (BE4), and HA tag (not shown).

### Yeast Growth Assay

To compare yeast growth on agar plates, equal numbers of cells (5 μl of cellular suspension with OD₆₀₀=2) were spotted on agar plates, and incubated at 30° C. for 3 days (complex medium), or 7 days (adenine deficient medium, −Ade). The desirable color saturation on complex medium was achieved by incubating the plates for 3 additional days at 4° C.

### Immunoblotting

To obtain cell lysates, cells grown in 50 ml of liquid medium to late logarithmic stage were pelleted, washed with water, resuspended in a 50 mM Tris pH 7.6 buffer containing 50 mM KCl, 10 mM MgCl₂, 5% glycerol, 10 mM PMSF, and an anti-protease cocktail for yeast (Sigma) 1:100, and lysed by vortexing with glass beads. Cell debris was removed by centrifugation at 4° C. for 5 min at 10,000 g. Protein concentration was measured by the Bradford reagent from BioRad [60].

To visualize SDS-stable oligomers of AβMRF by SDS electrophoresis in polyacrylamide or agarose gels, equal amounts of lysate proteins were treated with sample buffer (50 mM Tris/HCl pH 6.8 for acrylamide, or 25 mM Tris 200 mM glycine for agarose gels, respectively) containing 1% SDS for 7 min at room temperature. Oligomers of AβMRF were also able to withstand 2% SDS treatment at room temperature (not shown). To disaggregate AβMRF oligomers into monomers, lysates were boiled for 5 min in sample buffer supplemented with 2% SDS and 2% β-mercaptoethanol (not shown).

SDS-treated lysates were resolved by SDS-electrophoresis in 7.5% polyacrylamide gels as described [61], and transferred to an Immun-Blot PVDF membrane (Bio-Rad). Immunodetection was performed using monoclonal antibodies against Sup35p's RF domain (BE4, developed by Dr. V. Prapapanich in our laboratory), monoclonal antibodies against Aβ₁₋₁₇ (6E10, from Signet Laboratories), or anti-oligomer antibodies (a kind gift from Drs. R. Kayed and C. Glabe; [44]). Signal was revealed using a Western-Star chemiluminescence development kit (Applied Biosystems) as suggested by the manufacturer. Molecular weight standards were treated in the same sample buffer as the experimental samples, and were revealed after immunodetection by staining the membrane with the Coomassie Brilliant Blue R-250 reagent. The position of the 650 kDa molecular weight marker was calculated using AlphaEaseFC software.

For better resolution of the AβMRF oligomers, we used SDS electrophoresis in agarose as described elsewhere [62], with the following changes. The SDS-treated lysates (see above) were electrophoretically separated in horizontal 1.5% agarose gels in a 25 mM Tris buffer containing 200 mM glycine and 0.1% SDS. Proteins were transferred onto a PVDF membrane in a 25 mM Tris buffer containing 200 mM glycine, 15% methanol, 0.1% SDS using a semi-dry blotting unit FB-SDB-2020 (Fisher Scientific) at 1 mA per cm² of the gel/membrane surface for 1 hr, and processed as described above. Densitometry was performed using Alpha Imager 2200 (Alpha Innotech) and processed on AlphaEaseFC imaging software.

To confirm equal protein loading, we first determined protein concentrations in the lysates by Coomassie Brilliant Blue (Bradford reagent). We then brought the protein concentration in all samples to the same value, and in the same volume, followed by an additional verification by Bradford reagent. After the immunodetection, the membrane was stained with Coomassie to confirm equal protein loading.

### Aβ42 Polymerization and Immunoblotting

Recombinant Aβ42 peptide (powdered Aβ₄₂-acetate from Rpeptide) was polymerized according to the manufacturer's suggestions. Briefly, a 1 mg/ml solution of Aβ₄₂ was made by resuspending 0.5 mg of Aβ₄₂ powder in 100 μl of 2.5 mM NaOH and adding 400 μl of phosphate buffered saline solution. Polymerization proceeded at room temperature with constant rotation (60 rpm). Polymerization was measured by Thioflavin T fluorescence (λ_{EX}=442 nm; μ_{EM}=483 nm). To perform immunoblotting, a sample containing 5 μg of polymerized Aβ42 was treated with 1% SDS and resolved by SDS electrophoresis in 1.5% agarose and processed as described above.

### Immunoprecipitation

Samples (500 μl) containing 800 μg of total lysate proteins were incubated with 6 μl of anti-HA antibodies immobilized on agarose beads using a Pro-Found HA-Tag Co-IP kit (Pierce), for 1.5 hrs at 4° C. Following incubation, the beads were washed three times with 0.5 ml of phosphate buffered saline containing 0.05% Tween 20 to remove the non-specifically bound proteins. Immunoprecipitated protein complexes were eluted with hot (95° C.) 0.3 M Tris buffer pH 6.8 containing 5% SDS, resolved by electrophoresis in 10% polyacrylamide gels, and analyzed by immunoblotting using monoclonal antibodies against the RF domain or against Hsp104 (SPA-1040, from Stressgen).

### LIST OF ABBREVIATIONS

AD, Alzheimer's disease; Aβ, amyloid-(β protein; RF, release factor; SDS, sodium dodecylsulfate; Hsp, heat shock protein; YPD, yeast extract/peptone/dextrose; HA, human influenza hemagglutinin; PCR, polymerase chain reaction; PMSF, phenylmethylsulfonylfluoride; PVDF, polyvinylidenfluoride; Gu, guanidine hydrochloride; WT, wild type; sHsp, small heat shock protein.

### LIST OF REFERENCES

1. LaFerla F M, Oddo S: Alzheimer's disease: Abeta, tau and synaptic dysfunction. Trends Mol Med 2005, 11:170-176.
2. Lesne S, Koh M T, Kotilinek L, Kayed R, Glabe C G, Yang A, Gallagher M, Ashe K H: A specific amyloid-beta protein assembly in the brain impairs memory. Nature 2006, 440:352-357.
3. Hardy J, Selkoe D J: The amyloid hypothesis of Alzheimer's disease: progress and problems on the road to therapeutics. Science 2002, 297:353-356.
4. Walsh D M, Selkoe D J: Deciphering the molecular basis of memory failure in Alzheimer's disease. Neuron 2004, 44:181-193.
5. Klein W L, Stine W B, Jr., Teplow D B: Small assemblies of unmodified amyloid beta-protein are the proximate neurotoxin in Alzheimer's disease. Neurobiol Aging 2004, 25:569-580.
6. LeVine H, 3rd, Scholten J D: Screening for pharmacologic inhibitors of amyloid fibril formation. Methods Enzymol 1999, 309:467-476.
7. Soto C, Estrada L: Amyloid inhibitors and beta-sheet breakers. Subcell Biochem 2005, 38:351-364.
8. Fisher A C, Kim W, DeLisa M P: Genetic selection for protein solubility enabled by the folding quality control feature of the twin-arginine translocation pathway. Protein Sci 2006, 15:449-458.
9. Sherman M Y, Muchowski P J: Making yeast tremble: yeast models as tools to study neurodegenerative disorders. Neuromolecular Med 2003, 4:133-146.
10. Mager W H, Winderickx J: Yeast as a model for medical and medicinal research. Trends Pharmacol Sci 2005, 26:265-273.
11. Outeiro T F, Lindquist S: Yeast cells provide insight into alpha-synuclein biology and pathobiology. Science 2003, 302:1772-1775.
12. Willingham S, Outeiro T F, DeVit M J, Lindquist S L, Muchowski P J: Yeast genes that enhance the toxicity of a mutant huntingtin fragment or alpha-synuclein. Science 2003, 302:1769-1772.
13. Bach S, Talarek N, Andrieu T, Vierfond J M, Mettey Y, Galons H, Dormont D, Meijer L, Cullin C, Blondel M: Isolation of drugs active against mammalian prions using a yeast-based screening assay. Nat Biotechnol 2003, 21:1075-1081.
14. Derkatch I L, Uptain S M, Outeiro T F, Krishnan R, Lindquist S L, Liebman S W: Effects of Q/N-rich, polyQ, and non-polyQ amyloids on the de novo formation of the [PSI+] prion in yeast and aggregation of Sup35 in vitro. Proc Natl Acad Sci U S A 2004, 101:12934-12939.
15. Rochet J C, Outeiro T F, Conway K A, Ding T T, Volles M J, Lashuel H A, Bieganski R M, Lindquist S L, Lansbury P T: Interactions among alpha-synuclein, dopamine, and biomembranes: some clues for understanding neurodegeneration in Parkinson's disease. J Mol Neurosci 2004, 23:23-34.
16. Zabrocki P, Pellens K, Vanhelmont T, Vandebroek T, Griffloen G, Wera S, Van Leuven F, Winderickx J: Characterization of alpha-synuclein aggregation and synergistic toxicity with protein tau in yeast. Febs J 2005, 272:1386-1400.
17. Vandebroek T, Vanhelmont T, Terwel D, Borghgraef P, Lemaire K, Snauwaert J, Wera S, Van Leuven F, Winderickx J: Identification and isolation of a hyperphosphorylated, conformationally changed intermediate of human protein tau expressed in yeast. Biochemistry 2005, 44:11466-11475.
18. Gokhale K C, Newnam G P, Sherman M Y, Chemoff Y O: Modulation of prion-dependent polyglutamine aggregation and toxicity by chaperone proteins in the yeast model. J Biol Chem 2005, 280:22809-22818.
19. Wickner R B, Liebman, S. W., Saupe, S. J.: Chapter 7: “Prions of Yeast and Filamentous Fungi: [URE3], [PSI⁺], [PIN⁺], and [Het-s]”. In Prion Biology and Diseases. Second edition. Edited by Prusiner S B: Cold Spring Harbor Laboratory Press, Cold Spring Harbor, N.Y.; 2004: 305-372
20. Chien P, Weissman J S, DePace A H: Emerging principles of conformation-based prion inheritance. Annu Rev Biochem 2004, 73:617-656.
21. Wickner R B, Edskes H K, Ross E D, Pierce M M, Baxa U, Brachmann A, Shewmaker F: Prion genetics: new rules for a new kind of gene. Annu Rev Genet. 2004, 38:681-707.
22. Satpute-Krishnan P, Serio T R: Prion protein remodelling confers an immediate phenotypic switch. Nature 2005, 437:262-265.
23. Sanchez Y, Lindquist S L: HSP104 required for induced thermotolerance. Science 1990, 248:1112-1115.
24. Lindquist S, Kim G: Heat-shock protein 104 expression is sufficient for thermotolerance in yeast. Proc Natl Acad Sci USA 1996, 93:5301-5306.
25. Chemoff Y O, Lindquist S L, Ono B, Inge-Vechtomov S G, Liebman S W: Role of the chaperone protein Hsp104 in propagation of the yeast prion-like factor [psi+]. Science 1995, 268:880-884.
26. Kryndushkin D S, Alexandrov I M, Ter-Avanesyan M D, Kushnirov V V: Yeast [PSI+] prion aggregates are formed by small Sup35 polymers fragmented by Hsp104. J Biol Chem 2003, 278:49636-49643.
27. Shorter J, Lindquist S: Hsp104 catalyzes formation and elimination of self-replicating Sup35 prion conformers. Science 2004, 304:1793-1797.
28. Wegrzyn R D, Bapat K, Newnam G P, Zink A D, Chemoff Y O: Mechanism of prion loss after Hsp104 inactivation in yeast. Mol Cell Biol 2001, 21:4656-4669.
29. Ness F, Ferreira P, Cox B S, Tuite M F: Guanidine hydrochloride inhibits the generation of prion “seeds” but not prion protein aggregation in yeast. Mol Cell Biol 2002, 22:5593-5605.
30. Grimminger V, Richter K, Imhof A, Buchner J, Walter S: The prion curing agent guanidinium chloride specifically inhibits ATP hydrolysis by Hsp104. J Biol Chem 2004, 279:7378-7383.
31. Tuite M F, Mundy C R, Cox B S: Agents that cause a high frequency of genetic change from [psi+] to [psi−] in Saccharomyces cerevisiae. Genetics 1981, 98:691-711.
32. Serio T R, Cashikar A G, Moslehi J J, Kowal A S, Lindquist S L: Yeast prion [psi+] and its determinant, Sup35p. Methods Enzymol 1999, 309:649-673.
33. Chemoff Y O, Uptain S M, Lindquist S L: Analysis of prion factors in yeast. Methods Enzymol 2002, 351:499-538.
34. Cox B S: [PSI], a cytoplasmic suppressor of super-suppression in yeast. Heredity 1965, 20:505-521.
35. Williams A D, Portelius E, Kheterpal I, Guo J T, Cook K D, Xu Y, Wetzel R: Mapping abeta amyloid fibril secondary structure using scanning proline mutagenesis. J Mol Biol 2004, 335:833-842.
36. Hilbich C, Kisters-Woike B, Reed J, Masters C L, Beyreuther K: Substitutions of hydrophobic amino acids reduce the amyloidogenicity of Alzheimer's disease beta A4 peptides. J Mol Biol 1992, 228:460-473.
37. Morimoto A, Irie K, Murakami K, Masuda Y, Ohigashi H, Nagao M, Fukuda H, Shimizu T, Shirasawa T: Analysis of the secondary structure of beta-amyloid (Abeta42) fibrils by systematic proline replacement. J Biol Chem 2004, 279:52781-52788.
38. Palmert M R, Podlisny M B, Witker D S, Oltersdorf T, Younkin L H, Selkoe D J, Younkin S G: The beta-amyloid protein precursor of Alzheimer disease has soluble derivatives found in human brain and cerebrospinal fluid. Proc Natl Acad Sci USA 1989, 86:6338-6342.
39. Podlisny M B, Walsh D M, Amarante P, Ostaszewski B L, Stimson E R, Maggio J E, Teplow D B, Selkoe D J: Oligomerization of endogenous and synthetic amyloid beta-protein at nanomolar levels in cell culture and stabilization of monomer by Congo red. Biochemistry 1998, 37:3602-3611.
40. Walsh D M, Tseng B P, Rydel R E, Podlisny M B, Selkoe D J: The oligomerization of amyloid beta-protein begins intracellularly in cells derived from human brain. Biochemistry 2000, 39:10831-10839.
41. Walsh D M, Selkoe D J: Oligomers on the brain: the emerging role of soluble protein aggregates in neurodegeneration. Protein Pept Lett 2004, 11:213-228.
42. Allen K D, Wegrzyn R D, Chernova T A, Muller S, Newnam G P, Winslett P A, Wittich K B, Wilkinson K D, Chemoff Y O: Hsp70 chaperones as modulators of prion life cycle: novel effects of Ssa and Ssb on the Saccharomyces cerevisiae prion [PSI+]. Genetics 2005, 169:1227-1242.
43. Bagriantsev S N, Kushnirov V V, Liebman S W: Analysis of amyloid aggregates using agarose gel electrophoresis. Methods Enzymol 2006, 412: 33-48 (in press).
44. Kayed R, Head E, Thompson J L, McIntire T M, Milton S C, Cotman C W, Glabe C G: Common structure of soluble amyloid oligomers implies common mechanism of pathogenesis. Science 2003, 300:486-489.
45. Schirmer E C, Lindquist S: Interactions of the chaperone Hsp104 with yeast Sup35 and mammalian PrP. Proc Natl Acad Sci USA 1997, 94:13932-13937.
46. Glover J R, Lindquist S: Hsp104, Hsp70, and Hsp40: a novel chaperone system that rescues previously aggregated proteins. Cell 1998, 94:73-82.
47. Cashikar A G, Duennwald M, Lindquist S L: A chaperone pathway in protein disaggregation. Hsp26 alters the nature of protein aggregates to facilitate reactivation by Hsp104. J Biol Chem 2005, 280:23869-23875.
48. Haslbeck M, Miess A, Stromer T, Walter S, Buchner J: Disassembling protein aggregates in the yeast cytosol. The cooperation of Hsp26 with Ssa1 and Hsp104. J Biol Chem 2005, 280:23861-23868.
49. Muchowski P J, Wacker J L: Modulation of neurodegeneration by molecular chaperones. Nat Rev Neurosci 2005, 6:11-22.
50. Parsell D A, Kowal A S, Singer M A, Lindquist S: Protein disaggregation mediated by heat-shock protein Hsp104. Nature 1994, 372:475-478.
51. Bukau B, Horwich A L: The Hsp70 and Hsp60 chaperone machines. Cell 1998, 92:351-366.
52. Muchowski P J, Schaffar G, Sittler A, Wanker E E, Hayer-Hartl M K, Hartl F U: Hsp70 and hsp40 chaperones can inhibit self-assembly of polyglutamine proteins into amyloid-like fibrils. Proc Natl Acad Sci USA 2000, 97:7841-7846.
53. Krobitsch S, Lindquist S: Aggregation of huntingtin in yeast varies with the length of the polyglutamine expansion and the expression of chaperone proteins. Proc Natl Acad Sci USA 2000, 97:1589-1594.
54. Chemoff Y O, Derkach I L, Inge-Vechtomov S G: Multicopy SUP35 gene induces de-novo appearance of psi-like factors in the yeast Saccharomyces cerevisiae. Curr Genet. 1993, 24:268-270.
55. Nakayashiki T, Ebihara K, Bannai H, Nakamura Y: Yeast [PSI+] “prions” that are crosstransmissible and susceptible beyond a species barrier through a quasi-prion state. Mol Cell 2001, 7:1121-1130.
56. Crist C G, Nakayashiki T, Kurahashi H, Nakamura Y: [PHI+], a novel Sup35-prion variant propagated with non-Gln/Asn oligopeptide repeats in the absence of the chaperone protein Hsp104. Genes Cells 2003, 8:603-618.
57. Sherman F: Getting started with yeast. Methods Enzymol 2002, 350:3-41.
58. DePace A H, Santoso A, Hillner P, Weissman J S: A critical role for amino-terminal glutamine/asparagine repeats in the formation and propagation of a yeast prion. Cell 1998, 93:1241-1252.
59. Cormack B P, Valdivia R H, Falkow S: FACS-optimized mutants of the green fluorescent protein (GFP). Gene 1996, 173:33-38.
60. Bradford M M: A rapid and sensitive method for the quantitation of microgram quantities of protein utilizing the principle of protein-dye binding. Anal Biochem 1976, 72:248-254.
61. Laemmli U K: Cleavage of structural proteins during the assembly of the head of bacteriophage T4. Nature 1970, 227:680-685.
62. Bagriantsev S, Liebman S W: Specificity of Prion Assembly in Vivo: [PSI+] and [PIN+] form separate structures in yeast. J Biol Chem 2004, 279:51042-51048.

While the principals of this invention have been described in connection with specific embodiments, it should be understood clearly that these descriptions are added only by way of example and are not intended to limit, in any way, the scope of this invention. Other advantages and features of this invention will become apparent from the claims hereinafter, with the scope of those claims determined by their reasonable equivalents, as would be understood by those skilled in the art.

What is claimed is:

1. A method of screening for a compound that decreases aggregation of amyloidogenic proteins, wherein the method comprises (a) contacting one or more yeast cells with a candidate compound, wherein the yeast cells express a fusion protein comprising an amyloidogenic peptide and one or more domains of Sup35p necessary for translation termination and wherein the yeast cells express one or more marker proteins from alleles having one or more premature termination signals, under conditions that allow for aggregation of the fusion protein, wherein fusion protein aggregation is essential for yeast cell viability on media lacking the essential nutrient of the molecular pathway in which the marker protein is involved; (b) measuring the viability of the yeast cell on media lacking the essential nutrient against the viability of the yeast cell on the same media supplemented with the nutrient; (c) comparing the level of viability with the level of viability of a yeast cell not contacted with the candidate compound.

2. The method of claim 1 further comprising (d) wherein the color of the yeast colonies contacted with the candidate compound on complex medium is assayed and compared with the color of yeast colonies not contacted with the candidate compound, wherein the marker protein is Ade1p expressed from an ade1-14 allele.

3. The method of claim 1, wherein the amyloidogenic peptide is Aβ42.

4. The method of claim 1, wherein the aggregation of the amyloidogenic peptide results in a disease selected from the group consisting of Alzheimer's disease, Parkinson's disease, Familial Amyloid Polyneuropathy, a Tauopathy, Trinucliotide disease, transmissible spongiform encephalopathies (TSEs), Alzheimer's (AD), and Huntington's Disease (HD).

5. The method of claim 1, wherein the one or more domains of Sup35p is selected from the group consisting of the N-prion domain, the M-middle domain, and the RF-release factor domain.

6. The method of claim 5, wherein the fusion protein consists of least one domain of the amyloid protein, the M-middle domain of Sup35p, and the RF-release factor domain of Sup35p.

7. The method of claim 6, wherein the fusion protein further consists of an HA tag between the M and RF domains.

8. The method of claim 1, wherein the one or more marker proteins is selected from the group consisting of ADE1, LYS2, LYS5, CAN1, MET2, MET15, GAL1, and URA3.

9. The method of claim 1, wherein the yeast cell is selected from the group consisting of Saccharomyces uvae, Saccharomyces kluyveri, Schizosaccharomyces pombe, Saccharomyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans.

10. A method of screening for a compound that decreases aggregation of amyloidogenic proteins, wherein the method comprises (a) contacting one or more yeast cells with a candidate compound wherein the yeast cells express a fusion protein comprising an amyloidogenic peptide and one or more domains of Sup35p necessary for translation termination and wherein the yeast cells express one or more marker proteins having one or more premature termination signals, under conditions that allow for aggregation of the fusion protein, wherein fusion protein aggregation causes growth inhibition on media containing an inhibitor of growth in the presence of the marker protein; (b) measuring the viability of the yeast cells on media not supplemented with growth inhibitor; (c) comparing the level of viability with the level of viability of a yeast cells not contacted with the candidate compound.

11. The method of claim 10, wherein the growth inhibitor is 5-FOA and the marker protein is Ura3p expressed from a ura3-14 allele.

12. The method of claim 10, wherein the aggregation of the amyloidogenic peptide results in a disease selected from the group consisting of Alzheimer's disease, Parkinson's disease, a Prion disease, Familial Amyloid Polyneuropathy, Trinucliotide disease, transmissible spongiform encephalopathies (TSEs), Alzheimer's (AD), Huntington's (HD), and Parkinson's Diseases.

13. The method of claim 10, wherein the one or more domains of Sup35p is selected from the group consisting of the N-prion domain, the M-middle domain, and the RF-release factor domain.

14. The method of claim 13, wherein the fusion protein consists of at least one domain of the amyloid protein, the M-middle domain of Sup35p, and the RF-release factor domain of Sup35p.

15. The method of claim 14, wherein the fusion protein further consists of an HA tag between the M and RF domains.

16. The method of claim 10, wherein the one or more marker proteins is selected from the group consisting of ADE1, LYS2, LYS5, CAN1, MET2, MET15, GAL1, and URA3.

17. The method of claim 10, wherein the yeast cell is selected from the group consisting of Saccharomyces uvae, Saccharomyces kluyveri, Schizosaccharomyces pombe, Saccharomyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp. and Geotrichum fermentans.

18. The method of claim 10, wherein the amyloidogenic peptide is Aβ42.

19. A kit useful for screening compounds that inhibit protein aggregation, wherein the kit comprises a yeast cell strain which expresses a fusion protein comprising amyloidogenic protein and one or more domains of Sup35p, wherein the yeast cell strain expresses one or more marker proteins having one or more termination signals, and wherein the yeast strain contains a genomic deletion of SUP35 (sup35Δ::LEU2).

20. The kit of claim 19, wherein the yeast cell strain further comprises a second genomic deletion of HSP104 (hsp104Δ::URA3).

21. The kit of claim 19, wherein the yeast cell strain further comprises the genomic background: MATa ade1-14 ura3-52 leu2-3,112 trp1-289 his3-200.

22. The kit of claim 19, wherein the amyloidogenic protein is Aβ42.
